(19) United States
(12) Patent Application Publication
(10) Pub. No.: US 2008/0188424 A1
(43) Pub. Date: Aug. 7, 2008

(54) TOPICAL NON-PHARMACEUTICAL COSMETIC, PERSONAL AND PET CARE PRODUCTS WITH CHIRAL TECHNOLOGY AND METHODS OF FORMULATING AND MANUFACTURING THE SAME

(76) Inventors: LINDA J. WALKER, Cardiff, CA (US); Darcy C. Taylor, Cardiff, CA (US)

Correspondence Address: Wright Law Group, PLLC;ATTN: Mark F. Wright, Suite 2, 7201 West Oakland, Chandler, AZ, 85226, US

(21) Appl. No.: 12/044,643

(22) Filed: Mar. 7, 2008

Related U.S. Application Data
(63) Continuation-in-part of application No. 11/776,060, filed on Jul. 11, 2007.
(60) Provisional application No. 60/807,021, filed on Jul. 11, 2006.

(51) Int. Cl. A61K 31/70; A61K 31/16; A61K 31/07; A61K 31/195; A61K 31/7012; A61K 31/01; A61Q 1/00

(52) U.S. Cl. 514/23; 514/613; 514/168; 514/563; 514/474; 514/762

(57) ABSTRACT

As disclosed herein, a series of compounds that are not generally intended for internalization such as cosmetic, skin and pet care products are formulated using chirally correct ingredients, organic ingredients, and natural or naturally derived ingredients. In this fashion, certain deleterious biological effects due to inadvertent or incidental internalization may be reduced or minimized to a greater extent. The chirally correct formulations may be used in various types of skin creams, cleansers, lotions, etc. that are formulated for application to the external surfaces of a biological entity, including humans and animals. In addition to the use of chirally correct ingredients, other formulations include additional natural and/or organic ingredients.

### CROSS REFERENCE TO RELATED APPLICATIONS

This application is a continuation-in-part of U.S. patent application Ser. No. 11/776,060, filed on Jul. 11, 2007, which application is now pending and which application claims the benefit of the filing date of U.S. Provisional Patent Application No. 60/807,021, filed on Jul. 11, 2006, all of which are incorporated herein by reference.

### BACKGROUND OF THE INVENTION

1. Field of the Invention

The present invention relates to the field of non-pharmaceutical cosmetic, personal and pet care products and more specifically relates to methods of formulation and manufacturing of products with chirally correct ingredients.

2. Background Art

In the world of pharmaceuticals, various compounds may be available in multiple forms, including as a racemate or as pure enantiomer, each of which might also have different potencies. The “chirality” or “handedness” of a given compound is described in chemistry by using the term “L” to define the left side of a molecular twin (the L-Isomer) and the term “D” to define the right side or the D-Isomer. While molecules of one “hand” are necessary to life, such as L-amino acids and D-sugars, scientists have learned that their mirror image or opposite “hand” will usually exhibit different characteristics, ranging from harmless to toxic or, in certain extreme cases, highly poisonous. Accordingly, current “best practice” standards for preparing compounds designed to be ingested by humans and animals dictate that both enantiomers be tested separately for their pharmacological effects. This is because many racemic compounds, when ingested by humans and/or animals, may have harmful effects and lead to undesired conditions such as cancer.

One compound may specifically interact with a cell receptor to produce the desired outcome, while the other compound might have no useful application or might have an unwanted effect through some other interaction.

It is estimated that chiral chemical demand in the USA will grow over nine percent annually through 2005. The fastest growing chiral end-uses will include pharmaceutical, non-pharmaceutical and cosmetic industries. The cosmetic and personal care industry use many raw materials and ingredients that are made to aid in the texture, feel, smell, look, performance and longevity of products, as well as to provoke a localized “therapeutic” response/change (hence the evolvement of the so-called “dermaceutical” and “cosmeceutical” grade products) as a means of relieving or eliminating various specified skin conditions.

However, even within the present standards for production of certain compounds such as pharmaceuticals, the effects of racemic compounds are not always fully considered. For example, many topically applied compounds such as skin creams and cleansers, skin lotions and suntan lotions are not presently manufactured with an eye towards ensuring chirally correct formulations. Since these compounds are not designed for ingestion or internalization, the chiral properties of these compounds have been generally downplayed or simply ignored.

Accordingly, it is possible that the use of manufactured ingredients includes with or added to these topically applied compounds such as vitamin A and vitamin D may be chirally incorrect. Exposure to these compounds via incidental or accidental internalization may lead to undesirable and potentially serious side effects, particularly as these chirally incorrect compounds accumulate in the body over time. In 1950's, with the introduction and widespread use of the chirally “incorrect” pharmaceutical known as Thalidomide. In 1979 when the chemistry of the drug was finally resolved from a chiral perspective, it was discovered that the left hand or L-form caused birth defects, while the right hand or D-form and the racemic DL-form both exhibited equal sedative ability. The effect of chiral fragrances on human autonomic nervous system parameters is also well documented. The efficiency of the precutaneous absorption of cosmetic products may also be influenced by the chirality of the ingredients.

The stratum corneum, the rate-limiting barrier in transdermal drug delivery, is chiral in nature and enantiomers behave differently with respect to their transport across the skin, resulting in enantioselective permeation. For example, in Metoprolol (a β1-selective adrenergic) biological activity of metoprolol resides entirely with the S-enantiomer while the R-enantiomer is therapeutically inactive. With regards to this, the area of most concern is the cosmetic and personal care industry. The cosmetic and personal care industries generally downplay their products and services by not taking the chiral nature of the ingredients in the formulation, which creates harmful effects in the case of accidentally or incidentally ingestion by humans and/or animals.

For example, lipstick is a commonly applied compound that is most certainly internalized, to one degree or another, by both women and men on a regular basis. While internalizing a small amount of lipstick is most likely relatively harmless, regardless of the racemic nature of the ingredients, the accumulation of racemic compounds over time may not be so benign. Similarly, pets such as dogs and cats may occasionally internalize topically applied compounds during their normal interaction with humans and the day-to-day grooming process that typically involves licking the skin.

Additionally, young children are known for their propensity to place all manner of objects into their mouths, including thumbs and other appendages, which may have been previously treated with a cream, lotion or other product containing chirally incorrect ingredients. Even though many of these compounds are tested and considered “safe” and “non-toxic,” that determination is not made on the basis of chirality but rather the known toxic effects associated with the chemical compounds, regardless of the chiral composition.

The non chiral harmful chemical compounds passage in to the skin was induced by various factors like dermal decontamination, artificial (e.g. chemically and/or physically induced) exfoliation, environmental damage and factors such as age, overall health, ethnicity, geography, etc. In addition, these compounds can also have a deleterious effect on the ability of the stratum corneum to act as a barrier to topically applied substances. Accordingly, the reliable and effective measure of biological protection offered by the skin has become an increasingly uncertain and potentially faulty system.

Human and animal bodies are being subjected to external agents of questionable safety and reactivity on a progressively expansive scale and those agents can exert undesirable biological responses not only locally in the skin, but also, when internalized, systemically throughout the entire body. Given the possibility of a compromised stratum corneum, coupled with the increasingly varied and complex nature of the products offered by the cosmetic and skin care industry, it may be useful to consider the structural knowledge of compounds being introduced to the body and the possible effects that a certain process for the formulation of these products may have on the biological functions of both human and animal organisms.

Accordingly, without improved processes and products that minimize the inadvertent, accidental and/or incidental internalization of chirally incorrect compounds, particularly over time, the deleterious effects of exposure to and internalization of chirally incorrect compounds may become more of a concern.

### BRIEF SUMMARY OF THE INVENTION

As disclosed herein, a series of compounds that are not generally intended for internalization such as cosmetic, skin and pet care products are formulated using chirally correct ingredients, organic ingredients, and natural or naturally derived ingredients. In this fashion, certain deleterious biological effects due to inadvertent or incidental internalization may be reduced or minimized to a greater extent. The chirally correct formulations may be used in various types of skin creams, cleansers, lotions, etc. that are formulated for application to the external surfaces of a biological entity, including humans and animals. In addition to the use of chirally correct ingredients, other formulations include additional natural and/or organic ingredients.

In a first aspect, the invention provides formulations that contain chirally correct components in non-pharmaceutical, cosmetic, personal and pet care products. A great variety of chirally correct bioactive substances may be included in the formulations according to the various preferred embodiments of the present invention.

In a second aspect, the invention comprise methods for preparing the chiral correct ingredient containing formulations in manufacturing non-pharmaceutical, cosmetic, personal and pet care products, the methods comprising applying effective amounts of the formulations to the invention to the skin.

### DETAILED DESCRIPTION OF THE PREFERRED EMBODIMENTS

The objective of this invention to provide a method of formulating and manufacturing non-pharmaceutical cosmetic, personal and pet care products with chiral technology for prevention of deleterious effects due to the accidental or incidental ingestion of achiral components in the above said products by humans and/or animals.

Descriptive Terminology Used in This Application. For purposes of further identifying and describing the various preferred embodiments of the present invention, the following terms and phrases shall have the meaning set forth below

“Undesirable biological response” is defined as any event in which normal bodily function is impaired due to a reaction to a single chemical or a compound of chemicals, in rapid or prolonged progression, in mild or severe manifestation due to accidental or incidental ingestion of achiral components by humans and/or animals.

Internalization” is defined as the movement of any external agent (natural or synthetic a chiral molecule) into the internal body. This includes primary pathways of internalization through the skin as well as secondary pathways of internalization such as consumption/ingestion and inhalation.

The “internal body” shall include every structure, system and organ involved in the biological responses that make possible normal bodily function, and includes (but is not limited to) the skin, vascular system, respiratory system, and digestive track. With regards to the skin, an agent will not be considered in the internal body until it has passed through the stratum corneum, which, due to its relatively inert and non-living composition, is not directly involved in normal bodily function.

“Reduce the risk” shall mean a reduction in the magnitude and severity of potential risks associated with the internalization of an achiral component in the non-pharmaceutical or food-grade products. Reduction of risk is an important concept, given that almost any agent available to biological organisms can be potentially harmful or toxic, depending on its usage, duration of exposure, and amount of exposure. Additionally, reduction of risk is not confined to a specific class or classification of agents, whether or not completely natural or synthetic, as virtually all agents have a limit to their safety, either immediately or associated with intermittent, short-term, or with prolonged exposure.

It is estimated that by using a normal complement of body and skin care products, the average woman applies more than 200 chemicals to her skin each day. Furthermore, recent research has shown that as much 60% of these chemical ingredients are absorbed into the bloodstream. It has also been found that the number of people with eczema, allergies and skin complaints is on the rise, with these conditions most likely being aggravated by the structural nature of the ingredients in basic cosmetic and skincare products. The best way to prevent potentially harmful synthetic and natural chemicals from entering the body is to use only chiral correct ingredients in the and cosmetic and pet care products.

Several examples of the use of racemic compounds will offer additional background as to the practical implications of the current practices in the cosmetics and skin care industries. For example, 1-alpha bisabolol, also known as levomenol, is colorless viscous oil that is the primary constituent of the essential oil derived from German chamomile (Matricaria recutita). It is almost insoluble in water and glycerin, but well soluble in ethanol. Bisabolol is also known to have various anti-irritant, anti-inflammatory and anti-microbial properties. Bisabolol also has a weak sweet floral aroma and is used in various fragrances. It has also been used for hundreds of years in cosmetics because of its perceived skin healing properties. In laboratory synthesis, the molecule that matches the natural form of bisabolol is the left-handed molecule but the synthetic version of bisabolol, typically used in cosmetics, is a racemic mixture. This racemic mixture doesn't exhibit the real effect of the natural ingredient and thereby the chiral correct component of bisabolol is necessary in the cosmetic formulations.

Similarly, the natural form of vitamin E is the d-alpha tocopherol molecule. The laboratory-synthesized molecule that matches the naturally occurring form is also the right-handed (d) version. With many cosmetic and skin care products advertised as containing vitamin E, the inclusion of the non-natural, “racemic” molecule is common. This version is the dl tocopherol version and has been known to cause allergic reactions in some people. Only the split “d” molecule mirrors the natural version but it is common to see the racemic version used in various formulations for products designed for use by humans.

Panthenol, also known as Vitamin B5, comes in two enantiomers, the D and L versions. While only D-panthenol (dexpanthenol) is considered biologically active, both forms are known to have moisturizing properties. For cosmetic use, panthenol comes either in the D form, or as a racemic mixture containing both the D and L enantiomers (dl-panthenol). The racemic version is commonly used in the cosmetic and skin care industry for a variety of topically applied products.

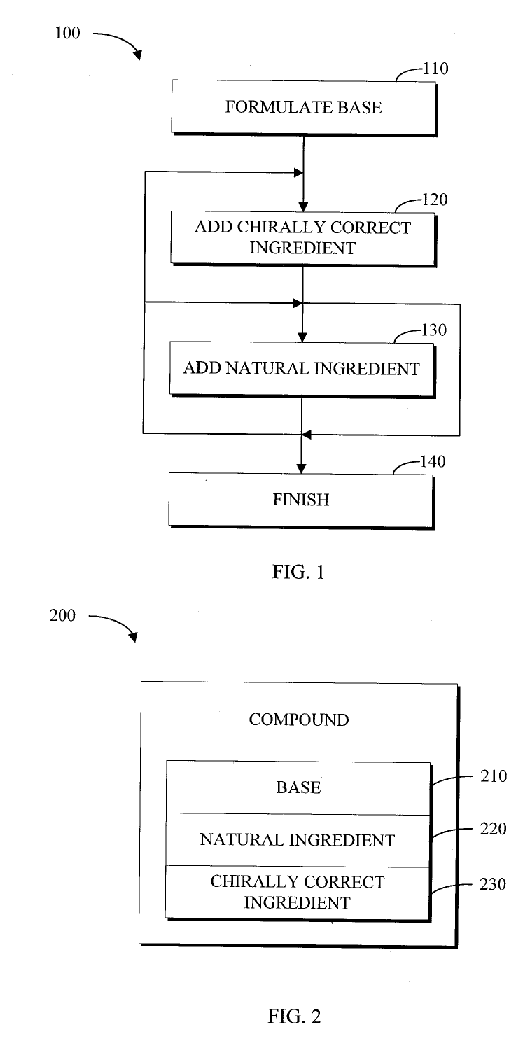

Referring now to FIG. 1, a flow chart for a method 100 of preparing a formulation in accordance with the preferred embodiments of the present invention is depicted. As shown in FIG. 1, after formulating a base (step 110), a chirally correct ingredient may be added to the base (step 120). Following this step, an organic ingredient may be added (step 130). As shown in FIG. 1, the steps of adding chirally correct and natural ingredients may be repeated as often as desired, depending on the specific application to be considered. Base 110 may be any type of base known to those skilled in the art and suitable for the applications described herein. The use a base 110 for topical applications such as a lotions, creams, cleansers, etc. is well known to those skilled in the art. Although the most preferred embodiments of the present invention will include the step of including a natural ingredient, this step may be omitted in certain applications and formulations.

Referring now to FIG. 2, a block diagram of a compound 200 in accordance with a preferred embodiment of the present invention is depicted. As shown in FIG. 2, the most preferred embodiments of compound 200 comprise a base 210, at least one natural ingredient 220 and at least one chirally correct ingredient 230. Those skilled in the art will recognize that the natural ingredient(s) 220 may be omitted in certain formulations.

Given the broad scope of the present invention, it would be useful to present several examples of specific formulations and methods of manufacture that may be considered illustrative but not limiting in the explanation of certain preferred embodiments of the present invention. The examples shown below are only two of the many products that could be formulated using the concepts and methods of the present invention.

The following examples are intended to further illustrate certain particularly preferred embodiments of the invention and are not intended to limit the scope of the invention in any way.

### EXAMPLE 1

Facial Cleanser Formulation. This is a formula and manufacturing process for a facial cleanser in accordance with a preferred embodiment of the present invention. Part A—In a glass beaker, bring 3220 gm. of treated water to a temperature of 80° centigrade. Add 0.1 gm. Biotin and 2 gm. of Aloe Powder and 1 gm. of Potassium Sorbate and 40 gm. Structure XL and 10 gm. of Methocel 10-101 Powder. Place Part A into a standard mixer on low speed.

Part B—Mix together 511 gm. Ricinoleamidopropyl Betaine with 439 gm. Ritafactant 138 AN (Deecyl Glucoside, Sodium Lauroyl Lactylate) and 5 gm. D-Panthenol and 11 gm. L-Bisabolol and 10.8 gm. D-Alpha Tocopherol. Add part B into part A while the mixer is on low speed.

Part C—Mix together 135.6 gm. Sorbitol with 1.2 gm. Octaquest E-30 and 30 gm. % STAIL (Phenethyl Alcohol 58% and Caprylyl Glycol 42% Conc.). Mix part C into previously combined part A and part B.

### EXAMPLE 2

Skin Lotion Formulation. This is a formula and manufacturing process for a skin lotion cleanser in accordance with a preferred embodiment of the present invention. Part A—Heat 3,250 ml of treated water to 85° centigrade and add 4 gm. L-Sodium Hyaluronate. After the Hyaluronate has dissolved, add 2 gm. Potassium Sorbate and mix well. Then, in a glass bowl add 80 ml Glycerin with 16 gm. Xanthan Gum to make a paste and combine with liquid ingredients.

Part B—Combine 320 gm. Oliven 1000 (B&T) with 371.6 gm. Myristol 318 (Caprylic/Capric Triglyceride) and 81 gm. Jojoba Oil. Heat combined ingredients to 85° centigrade.

Use a 4-inch dispersion blade and combine A with part B and mix together for 25 minutes. Follow this with a regular cool bath process and add the following ingredients at 40°-40 ml D-Vitamin E (D-Tocopherol)(Covitol IK00069) and 40 gm D-Panthenol and 20 gm L-Alpha Bisabolol and 0.2 gm Biotin and 50 ml Treated Water and 48 gm Stabil and 2 ml Octaquest E-30 (EDDS).

It is possible to include any and/or all of the various components, in any order, as may be required for the specific product formulation. Those skilled in the art will also recognize that, in at least some cases, the exact order of adding the various ingredients may be altered without materially affecting the outcome of the manufacturing process. Additionally, minor variations in temperature may be made for environmental conditions, altitude, etc. without departing from the spirit and scope of the present invention.

Accordingly, those skilled in the art will recognize that the unique combinations of ingredients set forth herein are selected so as to be the healthiest and chemically benign components possible for use in cosmetic and skin care applications. It should also be noted that the various preferred embodiments of the present invention are not directed towards preventing or eliminating all potential risks associated with internalization of racemic compounds. Rather, the methods of the present invention are directed towards reducing and minimizing toxicity or harmfulness, particularly with regards to long-term exposure due to inadvertent or accidental exposure to racemic compounds and agents that may produce undesired biological responses, as well as non-natural and non-organic compounds with unknown effect on the biological system of humans and animals.

Having described the invention with reference to particular compositions, theories of effectiveness, it will be apparent to those of skill in the art that it is not intended that the invention be limited by such illustrative embodiments or mechanisms, and that modifications can be made without departing from the scope or spirit of the invention, as defined by the appended claims. It is intended that all modifications and variations be included within the scope of the invention. The claims are meant to cover the claimed components and steps in any sequence which is effective to meet the objectives there intended, unless the context specifically indicates the contrary.

1. A method of formulating and manufacturing a non-pharmaceutical topically applied product, said method comprising the steps of:
creating a base for the skin care product;
adding at least one chirally correct ingredient to said base; and
adding at least one natural ingredient to said base.

2. The method of claim 1 wherein said at least one chirally correct ingredient is selected from a group consisting of sugars, vitamins, amino acids and organic acids.

3. The method of claim 2, wherein said at least one chirally correct ingredient comprises D-ribose.

4. The method of claim 3 wherein said D-ribose is present in a concentration range of 0.1 to 0.5%.

5. The method of claim 2 wherein said at least one chirally correct ingredient comprises D-panthenol and L-retinol.

6. The method of claim 5 wherein said D-panthenol and L-retinol are present in a concentration range of 0.1-0.8% and 0.1-1% respectively.

7. The method of claim 1 wherein said at least one chirally correct ingredient comprises L-serine, L-proline, D-proline and L-glutamine.

8. The method of claim 7 where said L-serine, L-proline, D-proline and L-glutamine are present in the concentrations of 0.01-0.02%, 0.01-0.03%, 0.01-0.02%, and 0.004-1% respectively.

9. The method of claim 1 wherein said at least one chirally correct ingredient comprises L-ascorbic acid, L-malic acid, and L-tartaric acid.

10. The method of claim 9 where said L-ascorbic acid, L-malic acid and L-tartaric acid are present in a range of concentration of 1-2.1%, 0.1-2%, and 0.1-2% respectively.

11. The method of claim 1 wherein said at least one natural ingredient comprises L-limonene, S-limonene, and L-alpha bisabolol.

12. The method of claim 11 where said L-limonene, S-limonene and L-alpha bisabolol are present in the concentrations of 0.1-1%, 0.05-1.25% and 0.01-2% respectively.

13. The method of claim 1 wherein said topically applied product comprises a topically applied product formulated for animals.

14. The method of claim 1 wherein said topically applied product comprises a topically applied cosmetic product for humans.

15. The method of claim 1 wherein said topically applied product comprises a topically applied skin care product for humans.

16. A non-pharmaceutical topically applied product, said product comprising:
a base;
at least one chirally correct component.

17. The non-pharmaceutical topically applied product of claim 16 wherein said at least one chirally correct component is selected from the group consisting of sugars, vitamins, amino acids and organic acids.

18. The non-pharmaceutical topically applied product of claim 16 further comprising at least one natural ingredient.

19. The non-pharmaceutical topically applied product of claim 18 wherein said at least one natural ingredient is selected from the group consisting of L-limonene, S-limonene and L-alpha bisabolol.

20. The non-pharmaceutical topically applied product of claim 18 wherein said at least one chirally correct component is selected from the group consisting of sugars, vitamins, amino acids and organic acids and wherein said at least one natural ingredient is selected from the group consisting of L-limonene, S-limonene and L-alpha bisabolol.
